(19) United States
(12) Patent Application Publication
(10) Pub. No.: US 2021/0161672 A1
(43) Pub. Date: Jun. 3, 2021

(54) SCAFFOLD WITH ADHESIVE FOR ARTICULAR CARTILAGE REPAIR

(71) Applicant: HISTOGENICS CORPORATION, Waltham, MA (US)

(72) Inventor: James KROM, Waltham, MA (US)

(73) Assignee: HISTOGENICS CORPORATION, Waltham, MA (US)

(21) Appl. No.: 16/973,987

(22) PCT Filed: Jun. 10, 2019
(86) PCT No.: PCT/US2019/036256

§ 371 Date: Dec. 10, 2020

Related U.S. Application Data
(60) Provisional application No. 62/683,358, filed on Jun. 11, 2018.

(51) Int. Cl. A61F 2/30

(52) U.S. Cl. CPC A61F 2/30756; A61F 2002/30766; A61F 2002/30761

(57) ABSTRACT

An injury or defect in articular cartilage is treated with a matrix implant that is applied above a barrier composition. The polymer-containing barrier composition is applied to the bottom of a cartilage lesion. The barrier composition can block migration of cells, blood, or other material from subchondral bone into the cartilage lesion.

### CROSS-REFERENCE TO RELATED APPLICATIONS

This application claims priority to U.S. Provisional Application 62/683,358, filed Jun. 11, 2018, the disclosure of which is herein incorporated by reference in its entirety.

### FIELD OF THE INVENTION

Matrix implants are implanted into an articular cartilage lesion above a barrier composition effective to inhibit the migration of cells, blood and other material from the subchondral area into the lesion.

### BACKGROUND

Articular cartilage consists of chondrocytes embedded in a large extracellular matrix comprised of water molecules, collagen fibers, and proteoglycans. Damage to the articular cartilage occurs in active individuals and older adults as a result of either acute or repetitive traumatic injury or aging. Such damage leads to pain, affects mobility, and can result in disability. There are many current therapeutic methods in use. Current surgical treatments include microfracture, lavage, debridement, drilling, and abrasion chondroplasty.

Lavage involves irrigation of the joint with solutions of sodium chloride, Ringer, or Ringer and lactate. Debridement involves smoothing out rough surfaces of cartilage and removing loose portions of the meniscus. These techniques provide temporary pain relief, but have little or no potential for further healing. The temporary pain relief is believed to result from removing degenerative cartilage debris, proteolytic enzymes and inflammatory mediators.

Microfracture involves the removal of damaged articular cartilage followed by physically insulting the underlying subchondral bone to exposed bone marrow and create bleeding. Microfracture is performed by drilling small holes into the subchondral bone to allow migration of bone marrow derived stem cells into the cartilage defect site. The surgery is performed by arthroscopy after cleaning the cartilage defect. The surgeon can use an awl to create a number of tiny fractures in the subchondral bone. Blood and bone marrow, which contains stem cells, seep out of the fractures and create a blood clot that releases cartilage-building cells. The body responds to microfracture as it would to an injury, which results in formation of new replacement cartilage. The blood clot introduces inflammatory cytokines, growth factors and mesenchymal stem cells (MSCs) to fill the defect. These agents, particularly the stem cells, allow for production of new cartilage.

Communication between repair tissue and the subchondral bone plate in a chondral lesion can facilitate chondral repair. Vasara, A. I. et al., OsteoArthritis and Cartilage, 2006, 14:1066-1074. Microfracture can provide for long-term improvement over 7-17 years. Steadman, J. R. et al., Arthroscopy, 2003, 9(5):477-484. At the same time, microfracture promotes formation of fibrocartilage rather than hyaline cartilage. Microfracture is also less effective in treating older patients, overweight patients, and patients with a cartilage lesion larger than 2.5 cm. These patients may have symptoms return only one to two years after surgery as the fibrocartilage wears away. At that point, such patients may have to reengage in articular cartilage repair surgery.

Other options include osteochondral autograft transplantation (OAT) and osteochondral allograft transplantation (OCA). OAT, however, is limited by donor site morbidity and the inability to treat large lesions, and OCA carries the risks of disease transmission and subchondral bone collapse. The direct transplantation of cells or tissue into a defect and the replacement of the defect with biologic or synthetic substitutions presently accounts for only a small percentage of surgical interventions.

### BRIEF DESCRIPTION OF THE DRAWINGS

FIG. 1 shows a histological analysis of implants above the subchondral bone, with a portion of the image in the upper panels magnified and shown in the lower panels. The left panels show tissue after a sutured cell construct was implanted above bone, in which an adhesive was applied below the cell construct. The middle panels show tissue after a cell construct was implanted above bone, in which an adhesive was applied below the cell construct. The right panels show tissue after a sutured cell construct was implanted above bone without adhesive.

### SUMMARY OF THE INVENTION

In one aspect is provided a method for treating an injury or defect in articular cartilage. The method comprises preparing a matrix implant, applying a barrier composition comprising a polymer to the bottom of the cartilage lesion, and implanting said implant above the applied barrier composition. In some embodiments, the barrier composition is applied to the subchondral bone.

In some embodiments, the barrier composition is effective to block migration of cells, blood, or other material from the subchondral area into the cartilage lesion.

In some embodiments, the matrix implant is an acellular matrix implant. In some embodiments, the acellular matrix implant comprises one or more of a Type I collagen, a Type II collagen, a Type IV collagen, a collagen containing proteoglycan, a collagen containing glycosaminoglycan, a collagen containing glycoprotein, a polymer of an aromatic organic acid, gelatin, agarose, hyaluronin, fibronectin, laminin, a bioactive peptide growth factor, a cytokine, elastin, fibrin, a polymer made of polylactic acid, a polymer made of polyglycolic acid, a poly(epsilon-caprolactone), a poly(vinyl alcohol), a poly(sebacic acid), poly(lactic-co-glycolic acid), poly(lactic acid-co-epsilon caprolactone), poly(lactic acid-co-vinyl alcohol), poly(lactic-co-sebacic acid), poly(glycolic acid-co-epsilon caprolactone), poly(glycolic acid-co-vinyl alcohol), poly(glycolic-co-sebacic acid), poly(epsilon-caprolactone-co-vinyl alcohol), poly(epsilon-caprolactone-co-sebacic acid), poly(vinyl alcohol-co-sebacic acid), a polyamino acid, a hydoxypolyamide, a polyamide, and a polypeptide gel. Exemplary hydroxypolyamides are described in U.S. Pat. Nos. 8,623,943; 9,315,624; and 9,505,882, all of which are incorporated by reference herein.

In some embodiments, the barrier composition comprises one or more of the following, or a polymerized product formed from the following: gelatin, Type I collagen, periodate-oxidized gelatin, a photo-polymerizable polyethylene glycol-co-poly(α-hydroxy acid) diacrylate macromer, 4-armed polyethylene glycols derivatized with N-(acyloxy)succinimide and thiol plus methylated collagen, a derivatized polyethylene glycol (PEG) cross-linked with alkylated collagen, tetra-N-hydroxysuccinimidyl or tetra-thiol derivatized PEG (e.g., SprayGel Adhesion Barrier System from Covidien, or CoSeal™ from Baxter Healthcare), and cross-linked PEG with methylated collagen.

In some embodiments, the barrier composition comprises a sealant. In some embodiments, the sealant forms a hydrogel after the barrier composition is applied to the subchondral bone.

In some embodiments, the barrier composition or the sealant comprises a polymer. In some embodiments, the polymer is gelatin, polyethylene glycol (PEG), a derivatized PEG, a poly(cyanoacrylate), a polyurethane, a poly(methylidene malonate), a polyvinyl alcohol, a polyamide, a hydroxypolyamide, a derivatized polyvinyl alcohol, an acrylic polymer, fibrin, gelatin, polystyrene with catechol side chains, a polyester, a polypeptide comprising dihydroxytyrosine, a poly(alpha-amino carboxylic acid) having catechol side chains, a polymer secreted by Phragmatopoma californica, a copolymer of polyethylene glycol and polylactide, a copolymer of polyethylene glycol and polyglycolide, a polyether, a polysaccharide, an oxidized polysaccharide, a polycation polyamine, a polyanion, a poly(ester urea), a copolymer of polyethylene glycol and poly-lactide or poly-glycolide, 4-armed pentaerythritol thiol and a polyethylene glycol diacrylate, 4-armed tetra-N-hydroxysuccinimidyl ester or a tetra-thiol derivatized PEG, a polymer formed from gelatin and oxidized starch, a polymer formed from photo-polymerizable polyethylene glycol-co-poly(a-hydroxy acid) diacrylate macromers, periodate-oxidized gelatin, serum albumin and di-functional polyethylene glycol derivatized with maleimidyl, succinimidyl, phthalimidyl and related active groups, and 4-armed polyethylene glycols derivatized with succinimidyl ester and thiol, and methylated collagen. In some embodiments, the polymer is gelatin or fibrin, and the barrier composition comprises thrombin or a crosslinking agent.

In some embodiments, the barrier composition comprises a component that modulates viscosity.

In some embodiments, the barrier composition comprises a stabilizer.

In some embodiments, the barrier composition comprises an enzyme effective to increase the rate of degradation of the barrier composition.

In some embodiments, the barrier composition further comprises a structural material. In some embodiments, the structural material comprises one or more of a fiber, fibrin, alginate, hyaluronic acid, gelatin, cellulose, or collagen.

In some embodiments, the method further comprises introducing a layer of a top protective biodegradable polymer above the matrix implant.

In some embodiments the matrix composition includes a component that enhances cell attachment and/or proliferation.

### DETAILED DESCRIPTION

### Definitions

Unless defined otherwise, technical and scientific terms used herein have the same meaning as commonly understood by one of ordinary skill in the art to which this invention belongs.

The terms “a,” “an,” and “the” do not denote a limitation of quantity, but rather denote the presence of “at least one” of the referenced item.

The term “cartilage”, as used herein, refers to a specialized type of connective tissue that contains chondrocytes embedded in an extracellular matrix. The biochemical composition of cartilage differs according to type, but in general comprises collagen, predominately type II cartilage along with other minor types (e.g., types IX and XI), proteoglycans, other proteins, and water. Several types of cartilage are recognized in the art, including, e.g., hyaline cartilage, articular cartilage, costal cartilage, fibrous cartilage, meniscal cartilage, elastic cartilage, auricular cartilage, and yellow cartilage.

The term “chondrocytes”, as used herein, refers to cells which are capable of producing components of cartilage tissue.

The term “support matrix” means biologically acceptable sol-gel or collagenous sponge, scaffold, honeycomb, hydrogel, a biologically acceptable material suitable for receiving activated migrating chondrocytes or osteocytes that provides a structural support for growth and three-dimensional propagation of chondrocytes and for formulating of new hyaline cartilage or for migration of osteochondrocytes into the bone lesions.

The inventors have found that formation of healthy hyaline cartilage rather than fibrocartilage is favored by depositing, into a cartilage lesion, a biodegradable acellular matrix implant above at least one layer of a barrier composition placed on the subchondral bone. Without wishing to be bound by theory, the barrier composition is effective to block migration of cells, blood or fluid from the subchondral area into the lesion, any of which may tend to promote fibrocartilage formation within the lesion. The inventors have found that the blockage could allow for chondrocytes derived from the implant, surrounding healthy cartilaginous tissue, and synovial stem cells resident in the synovial fluid or synovial membrane to develop the cartilage in the implant. Cells that migrate from the synovial membrane and other adjacent tissues may produce cartilaginous sulfated glycosaminoglycan. Sealants and adhesive components in the barrier composition can prevent penetration of the subchondral bone, prevent bone edema and allow the subchondral bone to heal fully in a manner independent of the implant. Bone edema is a source of pain and degeneration that leads to osteoarthritis. The methods described herein provide advantages over microfracture that include reducing risk of bone edema and promoting formation of hyaline cartilage over fibrocartilage.

### Matrix

A matrix implant for use in treating an injury or defect in articular cartilage is provided. The matrix implant is configured to be positioned above a barrier composition comprising a polymer that is applied to the bottom of a cartilage lesion, e.g., the subchondral bone.

In various embodiments, the matrix is a two or three-dimensional structural composition, or a composition able to be converted into a two or three-dimensional structure. In some embodiments, the matrix is a sponge-like structure or honeycomb-like lattice.

In some embodiments, the matrix is a support matrix. In some embodiments, the support matrix is prepared from one or more of Type I collagen, Type II collagen, Type IV collagen, gelatin, agarose, a collagen containing proteoglycans, glycosaminoglycans or glycoproteins, polymers of aromatic organic acids, fibronectin, laminin, bioactive peptide growth factors, cytokines, elastin, fibrin, polymers made of poly-acids such as polylactic, polyglycolic or polyamino acids, polycaprolactone, a polymer made of polylactic acid, a polymer made of polyglycolic acid, a poly(epsilon-caprolactone), a poly(vinyl alcohol), a poly(sebacic acid), poly(lactic-co-glycolic acid), poly(lactic acid-co-epsilon caprolactone), poly(lactic acid-co-vinyl alcohol), poly(lactic-co-sebacic acid), poly(glycolic acid-co-epsilon caprolactone), poly(glycolic acid-co-vinyl alcohol), poly(glycolic-co-sebacic acid), poly(epsilon-caprolactone-co-vinyl alcohol), poly(epsilon-caprolactone-co-sebacic acid), poly(vinyl alcohol-co-sebacic acid), a polyamino acid, a hydoxypolyamide, a polyamide, absorbable epsilon caprolactone polymer, polypeptide gel, copolymers thereof and combinations thereof. The gel solution matrix may be a polymeric thermo-reversible gelling hydrogel. The support matrix may have one or more of the following properties: biocompatibility, biodegradability, hydrophilicity, non-reactivity, a neutral charge, and a defined structure.

In some embodiments, the matrix is prepared by incubating, or entangling, a polysaccharide with a polyester comprising polylactic acid, polyglycolic acid, or a co-polymer comprising polylactic acid, polyglycolic acid, polyethylene glycol, polyvinyl alcohol, and poly(sebacic acid). The polysaccharide may be oxidized.

In some embodiments, the matrix comprises one or more of collagen, hyaluronan, and chondroitin sulfate.

In some embodiments, the barrier composition comprises one or more of the following, or a polymerized product formed from the following: gelatin, Type I collagen, periodate-oxidized gelatin, a photo-polymerizable polyethylene glycol-co-poly(α-hydroxy acid) diacrylate macromer, 4-armed polyethylene glycols derivatized with N-(acyloxy)succinimide and thiol plus methylated collagen, a derivatized polyethylene glycol (PEG) cross-linked with alkylated collagen, tetra-N-hydroxysuccinimidyl or tetra-thiol derivatized PEG (e.g., SprayGel Adhesion Barrier System from Covidien, or CoSeal™ from Baxter Healthcare), and cross-linked PEG with methylated collagen.

In some embodiments, the barrier composition comprises a sealant. In some embodiments, the sealant forms a hydrogel after the barrier composition is applied to the subchondral bone.

In some embodiments, the barrier composition or the sealant comprises a polymer. In some embodiments, the polymer is gelatin, polyethylene glycol (PEG), a derivatized PEG, a poly(cyanoacrylate), a polyurethane, a poly(methylidene malonate), a polyvinyl alcohol, a polyamide, a hydroxypolyamide, a derivatized polyvinyl alcohol, an acrylic polymer, fibrin, gelatin, polystyrene with catechol side chains, a polyester, a polypeptide comprising dihydroxytyrosine, a poly(alpha-amino carboxylic acid) having catechol side chains, a polymer secreted by Phragmatopoma californica, a copolymer of polyethylene glycol and polylactide, a copolymer of polyethylene glycol and polyglycolide, a polyether, a polysaccharide, an oxidized polysaccharide, a polycation polyamine, a polyanion, a poly(ester urea), a copolymer of polyethylene glycol and poly-lactide or poly-glycolide, 4-armed pentaerythritol thiol and a polyethylene glycol diacrylate, 4-armed tetra-N-hydroxysuccinimidyl ester or a tetra-thiol derivatized PEG, a polymer formed from gelatin and oxidized starch, a polymer formed from photo-polymerizable polyethylene glycol-co-poly(a-hydroxy acid) diacrylate macromers, periodate-oxidized gelatin, serum albumin and di-functional polyethylene glycol derivatized with maleimidyl, succinimidyl, phthalimidyl and related active groups, and 4-armed polyethylene glycols derivatized with succinimidyl ester and thiol, and methylated collagen. In some embodiments, the polymer is gelatin or fibrin, and the barrier composition comprises thrombin or a crosslinking agent.

In some embodiments the matrix composition includes a component that enhances cell attachment and/or proliferation.

In various embodiments, the matrix is a three-dimensional cell scaffold that comprises a biocompatible polymer formed from a plurality of fibers configured so as to form a non-woven three-dimensional open celled matrix. The open celled matrix may have a predetermined shape. The open celled matrix may have a predetermined pore volume fraction. The open celled matrix may have a predetermined pore shape. For example, the pores in the matrix may form a honeycomb lattice. The open celled matrix may have a predetermined pore size.

In various embodiments, the matrix or the support matrix has a defined pore size. Different pore sizes allow for faster or slower infiltration of the chondrocytes into the matrix, faster or slower growth and propagation of the cells and, ultimately, a higher or lower density of cells in a neo-cartilage construct, for example as described in U.S. Pat. No. 8,906,686, incorporated by reference herein. The pore size of the matrix may be adjusted by varying the pH of the gel solution, collagen concentration, and lyophilization conditions, for example. The pore size of the matrix may be from 50 to 500 μm, from 100 to 300 μm, or from 150 to 250 μm.

In various embodiments, the matrix may or may not be porous and can be applied as a caulk. Such matrix may comprise a polymeric thermo-reversible gelling hydrogel (TRGH). The caulk may be reconstituted using the patient's own synovial fluid, which may allow the matrix to be seeded with cells. Sponge-like materials could also be soaked with synovial fluid prior to implant. The matrix may be allowed to cure after application.

In some embodiments, the matrix comprises at least one therapeutic agent. The therapeutic agent can be, for example, an anti-infective agent, a pain medication, an analgesic, or anti-inflammatory agent, and an immunosuppressive agent.

### Barrier Composition

A barrier composition for use in treating an injury or defect in articular cartilage is provided. The barrier composition is applied to the bottom of a cartilage lesion, e.g., the subchondral bone. A matrix implant is positioned above a barrier composition. In some embodiments, a top protective biodegradable polymer is positioned above the matrix implant.

Deposition of a barrier composition onto the subchondral bone as described herein can allow for protection of the integrity of the lesion after cleaning during surgery, and can prevent migration of subchondral cells and cell products into the site of the cartilage defect. Without wishing to be bound by theory, prevention of such migration creates an environment for healthy hyaline cartilage to form, while also preventing formation of fibrocartilage by stem cells that migrate from bone marrow to the matrix via the subchondral bone.

The barrier composition may comprise a sealant. A sealant is a biologically acceptable typically rapid-gelling formulation having a specified range of adhesive and cohesive properties. The sealant may be a biologically acceptable rapidly gelling synthetic compound having adhesive and/or gluing properties. In various embodiments, the sealant is a hydrogel, such as derivatized polyethylene glycol (PEG), which is preferably cross-linked with a collagen compound, typically alkylated collagen. The hydrogel may form after the barrier composition is applied to the subchondral bone. Examples of sealants include, but are not limited to, tetra-N-hydroxysuccinimidyl or tetra-thiol derivatized PEG, or a combination thereof, commercially available from Cohesion Technologies, Palo Alto, Calif. under the trade name CoSeal™ (J. Biomed. Mater. Res Appl. Biomater., 58:545-555 (2001)); two-part polymer compositions that rapidly form a matrix where at least one of the compounds is polymeric, such as, polyamino acid, polysaccharide, polyalkylene oxide or polyethylene glycol and two parts are linked through a covalent bond (U.S. Pat. No. 6,312,725, herein incorporated by reference); and cross-linked PEG with methyl collagen, such as a cross-linked polyethylene glycol hydrogel with methyl-collagen. The sealant may gel or bond rapidly upon contact with tissue, particularly with subchondral bone.

In various embodiments, the barrier composition comprises a polymer. Exemplary polymers in the barrier composition include, but are not limited to, gelatin and oxidized starch, 4-armed penta-erythritol tetra-thiol and polyethylene glycol diacrylate, a polymer formed from photo-polymerizable polyethylene glycol-co-poly(a-hydroxy acid) diacrylate macromers, periodate-oxidized gelatin, serum albumin and di-functional polyethylene glycol derivatized with maleimidyl, succinimidyl, phthalimidyl and related active groups, and 4-armed polyethylene glycols derivatized with succinimidyl ester and thiol, and methylated collagen.

In some embodiments, the barrier composition comprises polyethylene glycol, a polyethylene glycol-based material, or a cross-linked polyethylene glycol. Exemplary polyethylene glycol (PEG)-based materials include, but are not limited to, CT-3, Coseal® (Baxter), Adherus® (Hyperbranch Medical Technology), and Resure® (Ocular Therapeutics). In some embodiments, the barrier composition comprises cross-linked polyethylene glycol and methylated collagen, e.g., CT-3. In some embodiments, the barrier composition is non-toxic to cells.

In some embodiments, the barrier composition comprises cyanoacrylate or a cyanoacrylate-based adhesive. Examples of cyanoacrylate-based adhesives include, but are not limited to, Dermabond® (Ethicon), Integuseal® (Kimberly Clark), Surgiseal® (Adhezion), Histoacryl® (Aesculap), Actabond™ (Bergen), and Indermil® (Covidien). Cyanoacrylates can bond to subchondral bone in the presence of water or moisture. Cyanoacrylates may have various chain lengths, which can affect the degree of binding and the biodegradability. In various embodiments, the barrier composition may be applied rapidly. In various embodiments, the cyanoacrylate or cyanoacrylate-based adhesive allows the barrier composition to resist infection.

In some embodiments, the barrier composition comprises polyurethane or a polyurethane-based adhesive. An example of a polyurethane-based adhesive includes, but is not limited to, TissuGlu® (Cohera). In some embodiments, the polyurethane and polyurethane-based adhesives have enhanced biodegradability, e.g., by modifying castor oil with isophorone diisocyanate or by reacting polycaprolactone diol and hexamethylene diisocyanate. The polyurethane may be based on polycaprolactone diol.

In some embodiments, the barrier composition comprises poly(methylidene malonate) or a poly(methylidene malonate)-based adhesive. An example of a poly(methylidene malonate)-based adhesive includes, but is not limited to, Bondease® (Optmed). The barrier composition may be pasted onto the subchondral bone, or applied to the subchondral bone using an applicator. The barrier composition comprising poly(methylidene malonate) or a poly(methylidene malonate)-based adhesive may have a rapid drying time after the adhesive sets.

In some embodiments, the barrier composition comprises derivatized polyvinyl alcohol or derivatized polyvinyl alcohol-based materials. An example of a derivatized polyvinyl alcohol-based material is Aeriseal® (Pulmonx). The derivatized polyvinyl alcohol may be formulated as a hydrogel, such as by adding water. Such derivatized polyvinyl alcohol-based hydrogels may have similar properties to hyaline cartilage such that application of the barrier composition can contribute to reduced pain and improved joint function.

In some embodiments, the barrier composition comprises an acrylic or an acrylic-based material.

In some embodiments, the barrier composition comprises fibrin or fibrin-based sealants. Examples of fibrin-based sealants include, but are not limited to, Tisseel® (Baxter) and Evicel® (Ethicon). The barrier composition may form upon mixture of two separate compositions, e.g., a fibrinogen-based composition and a thrombin-based composition, in which fibrin is formed when mixed. Mixture and application may be facilitated by use of an applicator or syringe with two or more chambers. Fibrin-based sealants may have low toxicity compared to other types of sealants. Fibrin-based sealants may be more biodegradable and biocompatible than other types of sealants. In various embodiments, the fibrin-based sealants are sterilized to remove viruses and other pathogens. In various embodiments, the barrier composition comprising fibrin or fibrin-based sealant may be pasted onto, or sprayed onto, the exposed subchondral bone.

In some embodiments, the barrier composition comprises gelatin and thrombin, or a mixture of gelatin and thrombin. The barrier composition may further comprise fibrin. Examples of such barrier compositions include, but are not limited to, Surgiflo® (Ethicon) and Floseal® (Baxter). The barrier composition may form upon mixture of two separate compositions, e.g., a composition comprising flowable gelatin and fibrinogen and a composition comprising thrombin. Mixture and application may be facilitated by use of an applicator or syringe with two or more chambers. In some embodiments, 90% of the fibrin-based sealant degrades within eight weeks. In various embodiments, the fibrin-based sealants are sterilized to remove viruses and other pathogens. In various embodiments, the barrier composition may be pasted onto, or sprayed onto, the exposed subchondral bone.

In some embodiments, the barrier composition comprises albumin with one or more chemical crosslinking agents. The barrier composition may form upon mixture of two separate compositions, e.g., a composition comprising albumin and a composition comprising the chemical crosslinking agent, e.g., glutaraldehyde. Mixture and application may be facilitated by use of an applicator or syringe with two or more chambers. Examples of such barrier compositions include, but are not limited to, Bioglue® (Cryolife), Progel™ (Neomend), and Preveleak® (Mallinckrodt Pharma).

In various embodiments, the barrier composition comprises polymers described in any of U.S. Pat. Nos. 6,312,725 and 6,624,245, and in Wallace, D. G., et al., J. Biomed. Mater. Res., 2001, 58:545-555, Hill, A. et al., J. Biomed. Mater. Res., 2001, 58:308-312, and Wise, P. E. et al., The American Surgeon, 68:553-562 (2002), all hereby incorporated by reference. For example, the CT-3 polymer is described in U.S. Pat. No. 6,312,725.

In some embodiments, the barrier composition comprises polystyrene with catechol side chains, e.g., as described in U.S. Patent Publication No. 2009/0036611, which is incorporated herein by reference in its entirety.

In some embodiments, the barrier composition comprises a polyester-based sealant, or a polyester. An example of a polyester-based sealant is poly(glycerol sebacate acrylate), described in Mandavi et al., Proc. Natl. Acad. Sci. USA, 2008, vol. 105, p. 2307. To enhance adhesion, poly(glycerol-co-sebacate acrylate) may be molded in a pattern based on the adhesive surfaces found on gecko feet, as described by Mandavi et al.

In some embodiments, the barrier composition comprises sandcastle worm glue, e.g., as described in U.S. Patent Publication No. 2016/0206300. The sandcastle worm (Phragmatopoma californica) can synthesize a polymeric adhesive liquid that cures over several hours to form an adhesive. The sandcastle worm glue may comprise a polyphenolic protein.

In some embodiments, the barrier composition comprises a KRYPTONITE™ bone matrix product, described in U.S. Pat. No. 7,964,207, which is incorporated herein by reference in its entirety.

In some embodiments, the barrier composition comprises a polymer prepared from a gel comprising gelatin and oxidized starch that is formed by mixing aqueous solutions of gelatin and oxidized starch. The gel can bond to tissue through a reaction of aldehyde groups on starch molecules and amino groups on proteins of tissue. In some embodiments, the adhesive bond strength is about 100 N/m. In some embodiments, the elastic modulus is about 8×10⁶ Pa. The gelled sealant is degraded by enzymes that cleave the peptide bonds of gelatin and the glycosidic bonds of starch. In some embodiments, 90% of the barrier composition degrades in 14 days.

In some embodiments, the barrier composition comprises a polymer made from a copolymer of polyethylene glycol and polylactide or polyglycolide, further containing acrylate side chains and gelled by light, in the presence of some activating molecules.

In some embodiments, the barrier composition comprises a polymer that comprises a water-soluble polymeric region. Exemplary polymers include polyethers, for example, polyalkylene oxides such as polyethylene glycol (“PEG”), polyethylene oxide (“PEO”), polyethylene oxide-co-polypropylene oxide (“PPO”), co-polyethylene oxide block or random copolymers, and polyvinyl alcohol (“PVA”), poly(vinyl pyrrolidinone) (“PVP”), poly(amino acids), poly (saccharides), such as dextran, chitosan, alginates, carboxymethylcellulose, oxidized cellulose, hydroxyethylcellulose and/or hydroxymethylcellulose, hyaluronic acid, and proteins such as albumin, collagen, casein, and gelatin. The water-soluble regions (e.g., PEG) of the macromers can have an average molecular weight of from about 3,500 Daltons to about 40,000 Daltons (e.g., from about 3,500 Daltons to about 35,000 Daltons, or from about 3,500 Daltons to about 30,000 Daltons, or from about 3,500 Daltons to about 25,000 Daltons). In some embodiments, the PEG has an average molecular weight of from about 3,500 Daltons to about 20,000 Daltons (e.g., from about 3,500 to about 15,000 Daltons, or from about 3,500 Daltons to about 10,000 Daltons, or from about 3,500 Daltons to about 5,000 Daltons). For example, the PEG can have an average molecular weight of about 35,000 Daltons or about 25,000 Daltons. In some embodiments, the PEG can have an average molecular weight of from about 3,500 Daltons to about 40,000 Daltons. For example, the PEG can have an average molecular weight of about 25,000 Daltons. In other embodiments, the PEG can have an average molecular weight of about 35,000 Daltons.

In some embodiments, the barrier composition comprises a PEG-based material, e.g., Duraseal™ (Covidien), Coseal® (Cohesion Technologies), and AdvaSeal™ (Ethicon). Barrier compositions comprising PEG and barrier compositions comprising PEG-based materials may have high adhesion strength, biocompatibility with the subchondral bone, and flexibility.

The barrier composition may comprise a polycation polyamine and at least one polyanion, where the at least one biodegradable polycation polyamine comprises modified gelatin, such as described in U.S. Pat. No. 8,283,384, which is incorporated herein by reference in its entirety. In some embodiments, the gelatin is modified with ethylenediamine. In some embodiments, the polyanion is a polyphosphate compound.

The barrier composition may comprise a poly(ester urea) (PEU)-based adhesive comprising a PEU polymer backbone having one or more side chains comprising a phosphate group and a crosslinking agent comprising a divalent metal salt, such as described in International Patent Publication No. WO2017/189534, which is incorporated herein by reference in its entirety. In some embodiments, the divalent metal salt comprises a salt of calcium, magnesium, strontium, barium, zinc, or any combination of calcium, magnesium, strontium, barium and zinc.

In various embodiments, the barrier composition comprises chondroitin sulfate. The chondroitin sulfate may be modified to include functional groups, such as methacrylate groups and aldehyde groups. The chondroitin sulfate may be crosslinked to form a hydrogel, e.g., by UV crosslinking with a photoinitiator.

In various embodiments, the barrier composition comprises multiple different polymers, sealants and/or adhesives. The properties of each polymer, sealant, or adhesive present in the barrier composition may compensate for the advantages and disadvantages of other polymers, sealants or adhesives present. For example, the barrier composition may be formulated with two polymers, with one polymer having a higher rate of degradation and bioresorption but lower adhesion strength, as compared to the other polymer. Such barrier composition may have acceptable degradation, bioresorption and adhesion strength.

In various embodiments, the barrier composition may be in the form of a hydrogel. The hydrogel may be of sufficient thickness so that the barrier composition effectively blocks migration of cells, blood, debris, and fluids from the subchondral space. Exemplary hydrogels and components thereof are described in U.S. Pat. No. 7,009,034, which is incorporated herein by reference in its entirety. A hydrogel may be formed by crosslinking PEG with chitosan. Another exemplary hydrogel may be synthesized by forming thioester linkages between thiol residues of dendron and a PEG macromer.

In some embodiments, the barrier composition comprises an oxidized polysaccharide, e.g., dextran and/or chitosan. Dextran is a complex polysaccharide with some branched structures, and unlike chitosan, does not have reactive amino groups. Oxidized dextran reacts with chitosan hydrochloride to form a gel that can adhere to tissue. Oxidized polysaccharides may be crosslinked with various materials or mixed together. Exemplary oxidized dextran-derived sealants are described in: Balakrishnan, et al., Acta Biomater. 2017, vol 53, p. 343; Lisman, et al., J. Biomater. Appl. 2014, vol. 28, p 1386; Araki, et al., J. Torac. Cardiovasc Surg. 2007, v. 134, p. 1241. Exemplary chitosan-derived sealants are described in Hogue, et al. Mol. Pharm. 2017, vol. 14, p. 1218; Nie, et al. Carbohydr. Polym. 2013, vol 96, p. 342; Medina, et al., Otolaryngol. Head Neck Surg. 2012, vol. 147, p. 357. An example of a chondroitin sulfate-derived sealant is described by Elisseeff et al., Mil. Med. 2014, vol. 179, p. 686.

In various embodiments, the barrier composition comprises a component that modulates viscosity. Such components can include, for example, glycosaminoglycans (e.g., hyaluronic acid), carboxymethyl cellulose (CMC), diethylene glycol dimethyl ether (“DIGLYME”), dimethylformamide (“DMF”), dimethyl succinate, dimethyl glutarate, dimethyl adipate, dextran, dextran sulfate, polyvinylpyrrolidone (PVP), combinations thereof, and the like. Thickening agents which can be used to adjust the viscosity of the compositions of the present disclosure include polycyanoacrylates, polylactic acid, polyglycolic acid, lactic-glycolic acid copolymers, poly-3-hydroxybutyric acid, polyorthoesters, polyanhydrides, pectin, combinations thereof, and the like.

In various embodiments, the barrier composition comprises a stabilizer. Suitable stabilizers can include those which prevent premature polymerization such as quinones, hydroquinone, hindered phenols, hydroquinone monomethyl ether, catechol, pyrogallol, benzoquinone, 2-hydroxybenzoquinone, p-methoxy phenol, t-butyl catechol, butylated hydroxy anisole, butylated hydroxy toluene, t-butyl hydroquinone, combinations thereof, and the like. Suitable stabilizers can also include anhydrides, silyl esters, sultones (e.g., α-chloro-α-hydroxy-o-toluenesulfonic acid-γ-sultone), sulfur dioxide, sulfuric acid, sulfonic acid, sulfurous acid, lactone, boron trifluoride, organic acids, alkyl sulfate, alkyl sulfite, 3-sulfolene, alkylsulfone, alkyl sulfoxide, mercaptan, alkyl sulfide, combinations thereof, and the like. In some embodiments, an anhydride such as maleic anhydride, sebacic acid anhydride, and/or azelaic acid anhydride, can be used as a stabilizer. In other embodiments antioxidants such as Vitamin E, Vitamin K1, cinnamic acid, and/or flavanone can be used as stabilizers.

In various embodiments, the stabilizers are present in an amount from about 0.01 to about 10 percent by weight of the barrier composition. In some embodiments, the stabilizers are present in an amount from about 0.1 to about 2 percent by weight of the barrier composition.

In some embodiments, an enzyme may be added to the barrier composition to increase its rate of degradation. Suitable enzymes include, for example, peptide hydrolases such as elastase, cathepsin G, cathepsin E, cathepsin B, cathepsin H, cathepsin L, trypsin, pepsin, chymotrypsin, γ-glutamyltransferase (γ-GTP), and the like; sugar chain hydrolases such as phosphorylase, neuraminidase, dextranase, amylase, lysozyme, oligosaccharase, and the like; oligonucleotide hydrolases such as alkaline phosphatase, endoribonuclease, endodeoxyribonuclease, and the like. In some embodiments, where an enzyme is added, the enzyme may be included in a liposome or microsphere to control the rate of its release, thereby controlling the rate of degradation of the barrier composition.

In various embodiments, the barrier composition further comprises collagen type-1. Without wishing to be bound by theory, the collagen type-1 may allow for cell migration on the surface of the barrier composition and stimulate coagulation of any blood from the subchondral bone.

In various embodiments, the barrier composition is in a hydrated form.

In some embodiments, the barrier composition can allow the matrix implant to remain securely in the collagen lesion or defect after implantation. There may be no need for suturing the matrix implant.

The matrix system may be an acellular matrix. The acellular matrix may be a tissue that has been decellularized such that the nuclear and cellular components are removed from the structural extracellular matrix. The acellular matrix may be prepared from tissue, including organs or isolated parts of organs. Exemplary tissues include heart valves, small intestine submucosa, dermis, amniotic membrane, bladder, omentum, pericardium, ligament, blood vessel, and the like. In one embodiment, the tissue includes, but is not limited to omentum and dermis. In another embodiment, the tissue is dermis. The tissue may be obtained from various mammalian sources including but not limited to human, goat, porcine, bovine, ovine, equine and the like. The tissue may be decellularized by conventional techniques, including steps such as tissue preservation, decellularization, washing, decontamination and storage.

The acellular matrix layer can be obtained by splitting the acellular matrix into thin sheets having a thickness of typically from about 50 microns to about 200 microns.

The matrix may further comprise at least one growth factor, which can be an epithelial growth factor (EGF), a vascular endothelial growth factor (VEGF), a transforming growth factor-β (TGF-β), a bone morphogenetic protein (BMP), a growth differentiation factor, an anti-dorsalizing morphogenetic protein-1 (ADMP-1), a basic fibroblast growth factor (bFGF), an acidic fibroblast growth factor (aFGF) a hedgehog protein, an insulin-like growth factor, a platelet-derived growth factor (PDGF), an interleukin (IL), a colony-stimulating factor (CSF), and/or an activin. In addition, a matrix of these embodiments can further comprise a collagen.

In some embodiments, the matrix may be fastened to the subchondral bone. Examples of fastening include, but are not limited to, a staple, a dart, a pin, a screw, a suture, a glue or a tack. In other aspects, a prosthesis can be a prosthetic plate.

In some embodiments, the matrix further comprises at least one therapeutic agent. In various embodiments, a therapeutic agent can be, without limitation, an anti-infective agent, a pain medication, an analgesic, or anti-inflammatory agent, and an immunosuppressive agent.

In some embodiments, the anti-infective agent is an antibiotic such as gentamicin, dibekacin, kanendomycin, lividomycin, tobramycin, amikacin, fradiomycin, sisomicin, tetracycline, hydrochloride, oxytetracycline, hydrochloride, rolitetracycline, doxycycline hydrochloride, ampicillin, piperacillin, ticarcillin, cephalothin, cephaloridine, cefotiam, cefsulodin, cefinenoxime, cefinetazole, cefazolin, cefotaxime, cefoperazone, ceftizoxime, moxolactam, latamoxef, thienamycin, sulfazecin, azthreonam or a combination thereof.

In some embodiments, the pain medication or analgesic is morphine, a nonsteroidal anti-inflammatory (NSAID) drug, oxycodone, morphine, fentanyl, hydrocodone, naproxyphene, codeine, acetaminophen, benzocaine, lidocaine, procaine, bupivacaine, ropivacaine, mepivacaine, chloroprocaine, tetracaine, cocaine, etidocaine, prilocaine, procaine, clonidine, xylazine, medetomidine, dexmedetomidine, or a VR1 antagonist.

In some embodiments, the barrier composition has an adhesive strength in the range 20-400 gf/cm². In some embodiments, the barrier composition has an adhesive strength in the range from 20-100 gf/cm², 40-120 gf/cm², 60-150 gf/cm², 80-200 gf/cm², 100-300 gf/cm², 200-400 gf/cm², 20-40 gf/cm², 30-50 gf/cm², 40-60 gf/cm², 50-70 gf/cm², 60-80 gf/cm², 70-90 gf/cm², 80-100 gf/cm², 90-110 gf/cm², 100-120 gf/cm², 110-130 gf/cm², 120-150 gf/cm², 140-170 gf/cm², 160-200 gf/cm², 180-220 gf/cm², 200-240 gf/cm², 220-260 gf/cm², 240-280 gf/cm² 260-300 gf/cm², 280-320 gf/cm², 300-350 gf/cm², 320-370 gf/cm², or 350-400 gf/cm².

In various embodiments, one or both of the barrier composition and matrix can be injected or implanted into the site of the cartilage defect. In various configurations, a site in need of tissue growth can comprise, without limitation, dermis, a rotator cuff tendon, an Achilles tendon, a ligament such as an anterior cruciate ligament (ACL), a posterior cruciate ligament, (PCL), a medial collateral ligament, a lateral collateral ligament or a periodontal figment, a sphincter such as an anal sphincter, a urethral sphincter, an esophageal sphincter or an antral sphincter, herniated tissue such as an abdominal hernia, a Cooper's hernia, a diaphragmatic hernia, an epigastric hernia, a femoral hernia, an incisional hernia, an inguinal hernia, an intervertebral disc hernia, a Littre's hernia, an obturator hernia, a pantaloon hernia, a perineal hernia, a properitoneal hernia, a Richter's hernia, a sciatic hernia, a sliding hernia, a Spigelian hernia or an umbilical hernia, an intervertebral disc nucleus, an intervertebral disc annulus, periosteal tissue, neural tissue such as central nervous system tissue (including spinal cord tissue) and demyelinated neural tissue, a nerve tunnel such as a nerve tunnel traversing bone tissue, a mitral valve, a tricuspid valve, an aortic heart valve, a pulmonary heart valve, vascular tissue comprising a stent, stenotic cardiovascular tissue, costal cartilage, meniscus cartilage, epiglottic cartilage, laryngeal cartilage such as arytenoid cartilage, cricoid cartilage, cuneiform cartilage and corniculate cartilage, external ear cartilage, or auditory tube cartilage.

### EXAMPLES

The present invention is also described and demonstrated by way of the following examples. However, the use of these and other examples anywhere in the specification is illustrative only and in no way limits the scope and meaning of the invention or of any exemplified term. Likewise, the invention is not limited to any particular preferred embodiments described here. Indeed, many modifications and variations of the invention may be apparent to those skilled in the art upon reading this specification, and such variations can be made without departing from the invention in spirit or in scope. The invention is therefore to be limited only by the terms of the appended claims along with the full scope of equivalents to which those claims are entitled.

### Example 1

Swine are divided into at least two groups, with at least one control group present. Each test group has a barrier composition applied, with the control group not having a barrier composition applied. In all groups, a cartilage defect is created in the weight bearing region of the femoral medial condyle of the knee joint.

In each test group, a barrier composition is applied onto the subchondral bone. Multiple test groups can be created to test various barrier compositions. In all groups, the same matrix is applied after any barrier composition is applied. The conditions involving application of matrix and any top polymer barrier above the matrix should be identical among all test groups and the control group.

At one month, and at additional time periods, testing is undertaken to determine whether the barrier composition prevents migration of subchondral components, e.g., cells and fluids, into the cartilage lesion. Such testing can include histological analysis and assaying the extent of fibrocartilage formation.

Additional testing may be undertaken, such as an assessment of inflammation, histological grading, and measuring the rate and extent of improvement of the mobility of the animals after the surgery.

The barrier compositions providing the most optimal prevention of migration of subchondral components, the least inflammation, and highest relative formation of hyaline cartilage to fibrocartilage will then be further tested for application in the clinic to human patients and animal patients.

### Example 2

Scaffolds were prepared for testing in swine as follows. A honeycomb-shaped porous collagen sponge (5 mm in diameter and 1 mm in thickness, Koken, Tokyo, Japan) was soaked in 25 μl of cold 0.3% neutralized collagen solution (Vitrogen, Cohesion Tech, Palo Alto, Calif.), and then incubated at 37° C. for 1 hour. The neutralized collagen solution solidified to form an acellular scaffold composed of collagen gel within the sponge.

An engineered cell construct implanted with adhesive and sutures was prepared by harvesting a biopsy from the porcine articular cartilage, mincing the biopsy and then digesting it in 1.5 mg/ml collagenase (CLS 1, Worthington, Freehold, N.J.), dissolved in Ham's F-12 (F-12, Invitrogen) with 100 μg/ml penicillin and 100 unit/ml streptomycin (P/S, Invitrogen) on a rotator at 37° C. for 18 hours. Non-digested tissue was removed using a cell strainer (70 μm mesh, BD Biosciences, Franklin Lakes, N.J.). The isolated porcine articular chondrocytes (pACs) were rinsed twice with PBS by centrifugation at 1000 rpm for 10 minutes. Viable and dead cells were counted using a hemocytometer and the trypan blue exclusion method. The cell viability at each biopsy was more than 95%.

pACs were seeded to monolayer culture dishes (100 mm in diameter) and incubated in DMEM/F-12 supplemented with 10% fetal bovine serum (FBS, Invitrogen) and P/S at 37° C., 5% CO₂ in air for 5 days. Prior to seeding the pACs into the collagen gel/sponge scaffold, the pACs were harvested from the culture dishes with 0.05% trypsin-EDTA (Invitrogen). A solution of 0.3% pepsin-digested acid-soluble collagen from bovine skin (Cohesion, Palo Alto, Calif.) was neutralized with 1/10 volume of 10×PBS and 0.1N NaOH. 300,000 pACs suspended in 25 μl of this neutralized collagen solution were placed onto a Teflon-made dish (Saint-Gobain Performance Plastics, Courbevoie, France) to maintain cell suspension within a desired area due to high fluid surface tension. A round collagen sponge composed of honeycomb-shaped pores (5 mm in diameter and 1.5 mm thick, Koken, Tokyo, Japan) was placed onto the cell suspension and allowed to absorb the solution. These cell constructs were incubated for 1 hour at 37° C. to allow the collagen solution to solidify into a gel. Medium was then added to the dish.

After a 12-hour incubation in the medium, the cell constructs were transferred to a pressure-proof culture chamber attached to a bioreactor (TEP-1, PURPOSE, Shizuoka, Japan) and incubated with cyclic hydrostatic pressure (HP) at 0-0.5 MPa, 0.5 Hz and medium replenishment at 0.05 ml/min, 37° C., 5% CO₂ in air for seven days. Then, the cell constructs were then transferred to a conventional 12-well culture plate (each well containing one cell construct in 2 ml of medium) and incubated for an additional 14 days at atmospheric pressure, 37° C., and 5% CO₂ in air. The culture medium was changed twice a week. The cell constructs, including surrogates, were harvested at day 21 for implantation. Cell viability and cellularity of the surrogate constructs were evaluated histologically.

### Example 3

Five different engineered cell constructs were implanted into swine and their properties compared. The five constructs were a) an empty defect control (“Empty”), b) an acellular scaffold control implanted with adhesive and sutures (“Scaffold”), c) an engineered cell construct implanted with adhesive and sutures (“Cell-construct”), d) a engineered cell construct implanted with adhesive alone (“Adhesive-cell-construct”), and e) a engineered cell construct implanted with sutures alone onto subchondral bone (“Sutured-cell-construct”).

Two rounds of surgeries were conducted in the swine. The protocol for the animal study was approved by the institutional animal care and use committee of Charles River Laboratories (Worcester, Mass.). Sixteen castrated 12- to 15-month-old male swine weighing 30 to 45 kg (Micro-Yucatan, Charles River Laboratories) were acclimatized more than one week prior to the first surgery. Animal anesthesia was induced with intramuscular injections of 0.04 mg/kg atropine sulfate (Patterson, Devens, Mass.), 0.55 mg/kg butorphanol tartrate (Patterson), 1.5 mg/kg xylazine (VEDCO, St. Joseph, Mo.), and 20 mg/kg ketamin hydrogen chloride (VEDCO) and maintained with inhalation anesthesia using Isoflurane (Patterson). The right knee joint was opened anterolaterally and the patella was luxated medially to expose the trochlea and medial condyle.

During the first round of surgeries, cartilage pieces from the non-weight-bearing sites of the right knees were collected to produce the cell constructs, and two full-thickness cartilage defects were created at the weight-bearing sites. Some of the defects were left empty (to serve as the Empty) and others were implanted with the acellular scaffolds (to serve as the Scaffold). During the second round of surgeries, the engineered cell constructs were implanted into the surgically created defects at the weight-bearing sites of the left knees, four weeks after collecting the cartilage from the same animals.

In swine, eight knees had two Empties created in each, eight knees had two Scaffolds implanted in each, eight knees had two Cell-constructs implanted in each, four knees had two Adhesive-cell-constructs implanted in each, and four knees had two Sutured-cell-constructs implanted in each. At two weeks after each surgery for Empty, Scaffold, and Cell-construct implantation, arthroscopy was conducted to evaluate joint space, the defects, and the implants. Six months after the implantation of the engineered cell constructs (or at seven months after the biopsy for empty defect creation and acellular scaffold implantation), the animals were euthanized for histological evaluation of the treatment sites.

During the surgical procedure, a few pieces of cartilage tissue (biopsy) were harvested from the trochlea ridges. Approximately 40 mg of the biopsy was obtained and kept in Ca²⁺- and Mg²⁺-free Dulbecco's phosphate buffered saline (DPBS; Invitrogen, Carlsbad, Calif.) with P/S.

Two full-thickness defects that were 5 mm in diameter were created at the weight-bearing site of the medial and distal femoral condyle using a dermal punch (5 mm), a beaver blade and a curette, while being careful to avoid damage to the subchondral bone. Four animals were designated as the empty control, and four animals were designated as the acellular scaffold control group.

To implant the cell constructs, the left knee joint was opened anterolaterally and the patella was luxated medially to expose the femoral medial condyle. Two chondral defects (5 mm in diameter) were created on the condyle applying the same method that was used for the empty defect and acellular scaffold controls. The cell constructs were placed in the defects and sutured each construct with four absorbable and two non-absorbable colored sutures and covered the constructs with adhesive (CT-3). The patella was reduced and the wound was closed in layers with absorbable sutures (0 PDS-II). The animals were then allowed free cage activity.

For the Adhesive-cell-construct implantation, the cell constructs were placed in the defect coated with the adhesive and covered the cell constructs with the adhesive alone. Each construct was sutured with two non-absorbable colored sutures for arthroscopic confirmation. The Sutured-cell-constructs were placed in the defect without the adhesive, and each construct was sutured with four absorbable and two non-absorbable colored sutures.

In the right side of the knee of the animals in the acellular scaffold control group, two acellular scaffolds were implanted. Briefly, the acellular scaffolds were implanted into the defects with a polyethylene glycol (PEG)/collagen-based tissue adhesive (CT-3, Angiotech, Vancouver, Canada) and each scaffold was sutured with six stitches using four absorbable (8-0 Vicryl, Ethicon, Somerville, N.Y.) and two non-absorbable blue sutures (8-0 Proline, Ethicon) to serve as markers during the arthroscopic evaluation. Following the suturing, the surfaces of the implants were covered with the CT-3 adhesive. After the patella was reduced, the wound was closed in layers with absorbable sutures (0 PDS-II, polydioxanone, Ethicon).

The animals were housed individually in cages and allowed free cage activity. The floor in each cage was covered with a thick rubber sheet to prevent slipping and additional trauma to the knee joint after surgery.

Two weeks after each open knee surgery involving the creation of empty defect controls, the implantation of Scaffolds, the implantation of Cell-constructs, the implantation of Adhesive-cell-constructs, and the implantation of Suture-cell-constructs, the cartilage surface was evaluated arthroscopically to confirm that the construct securely remained within the defect. If any implant did not remain at the site, the histology sample was removed from the evaluation. Also, the observed defects were translucent white and identical in appearance to the adjacent cartilage's surface. In fact, the implanted acellular construct or the engineered construct without color-marked sutures were not found, suggesting that the adhesive and the constructs did not inhibit cell migration from adjacent tissues.

The effectiveness of surgical adhesive was analyzed by conducted macroscopic and histological evaluations on the Cell-construct, Adhesive-cell-construct, and Suture-cell-construct groups at six months after the engineered cell construct implantation (seven months after biopsy and acellular scaffold implantation). The animals used were euthanized prior to the necropsy.

The articular surface was evaluated where the empty defects control, the implanted acellular scaffolds, and the implanted engineered cell constructs were located based on gross anatomical findings: the visual characteristics of filling tissue in the defect, filling ratio, color, and surface integration with host tissue, as compared to surrounding host cartilage. Macroscopic images were recorded with a digital camera (Coolpix E-995, Nikon USA, N.Y.). The repaired cartilage was then harvested with subchondral bone and adjacent cartilage, fixed in 4% paraformaldehyde (JT Baker, Phillipsburg, N.J.), and dissolved in PBS (pH 7.4) for 7 days on a gentle rotator at 4° C. The fixed tissues were then decalcified in 5% formic acid and sodium citrate solution (Sigma-Aldrich) for one to two weeks and embedded in paraffin. 4-μm thick longitudinal serial sections were cut and then stained with either hematoxylin and eosin (H&E) or safranin 0-fast green.

In addition, immunostaining using a collagen type II antibody was performed. For immunohistochemical analysis, the sections were deparaffinized in xylene and rehydrated with graded ethanol and PBS. To efficiently expose epitopes, the sections were incubated in 700 U/ml bovine testicular hyaluronidase (Sigma) and 2 U/ml pronase XIV (Sigma) at 37° C. for 1 hour. The sections were then incubated in a polyclonal antibody to type II collagen (Southern Biotech, Birmingham, Ala.).

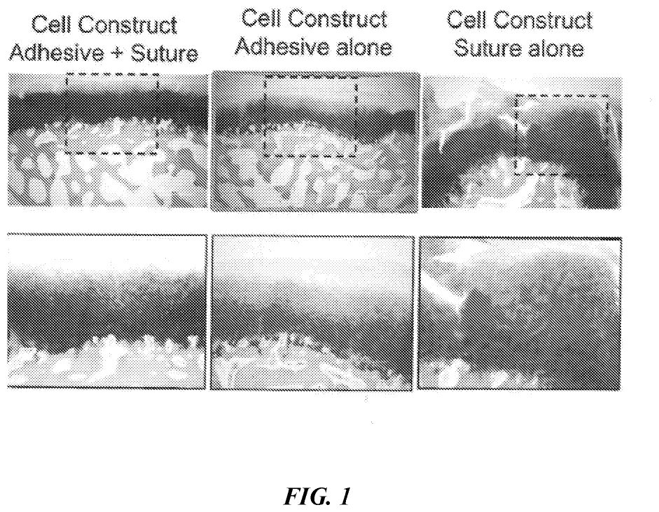

The histology data is shown in FIG. 1. The “Cell Construct Adhesive+Suture” and “Cell Construct Adhesive alone” groups had the adhesive barrier (CT-3 sealant) during surgery, while the “Cell Construct Suture alone” group did not have sealant applied during surgery. In the Cell Construct Adhesive+Suture” and “Cell Construct Adhesive alone” groups, there was a clear delineation of subchondral bone from the implant and healthy bone tissue appeared below the implant. In the “Cell Construct Suture alone” group, there was significantly greater penetration of the sutured cell implants into the bone as compared to the other groups.

Histological findings were then scored using a modified version of the histological grading scale developed by Sellers et al., J. Bone Joint Surg. Am., 1997, 79(10):1452-63. Three investigators blindly evaluated the longitudinal sections using the following criteria: 1) filling of the defect, 2) integration with host-adjacent cartilage, 3) matrix staining with Safranin O-fast green (metachromasia), 4) chondrocyte morphology, 5) architecture within the entire defect, 6) architecture of the surface, and 7) penetration. The Cell-construct group had adhesive and sutures, the Sutured-cell-construct group only had sutures, and the Adhesive-cell construct group only had adhesive. The scores are shown in Table 1 below.

TABLE 1

| | | Sutured-cell- | Adhesive-cell- |
|---|---|---|---|
| | | construct | construct |
| Classifications | Cell-construct | (n = 7) | (n = 8) |
| 1) Filling of defect | 3.0 | 3.0 | 2.9 ± 0.4 |
| 2) Integration with | 2.4 ± 0.6 | 2.8 ± 0.3 | 2.3 ± 0.7 |
| host adjacent cartilage | | | |
| 3) Matrix staining with | 2.6 ± 0.7 | 2.7 ± 0.6 | 2.8 ± 0.4 |
| Safranin O-fast green | | | |
| (metachromasia) | | | |
| 4) Chondrocytes | 2.4 ± 0.6 | 2.2 ± 1.0 | 2.4 ± 0.4 |
| morphology | | | |
| 5) Architecture | 2.5 ± 0.4 | 2.6 ± 1.1 | 2.8 ± 0.3 |
| within entire defect | | | |
| 6) Architecture | 2.5 ± 0.5 | 2.7 ± 0.4 | 2.5 ± 0.6 |
| of surface | | | |
| 7) Penetration | 2.7 ± 0.4^{a} | 1.7 ± 1.2^{a,b} | 2.9 ± 0.2^{b} |
| Total | 18 ± 1.6 | 17.7 ± 4.1 | 18.7 ± 1.5 |

Data are presented as mean ± SD.
^{a}p < 0.05 between indicated groups;
^{b}p < 0.05 between indicated groups

Regarding integration with adjacent cartilage in Table 1, the number of gaps or lack of continuity between the regenerated tissue and the adjacent cartilage was counted and classified. The regenerated tissue integrated with the Cell-constructs with a significantly smaller number of gaps than the gaps within the Empty (P<0.05). The integration of the Adhesive-constructs and of the Sutured-constructs was similar to the Cell-constructs in the number of gaps (Table 1).

Safranin O-fast green staining indicated the quality of sulfated cartilaginous matrix, which is a major component of articular cartilage. The regenerated tissues within the Scaffold and within the Cell-construct were slightly reduced in quantity compared to the adjacent cartilage. Matrix staining of the Adhesive-cell-constructs and of the Sutured-cell-constructs revealed that the quality of their sulfated cartilaginous matrix was similar to that of the Cell-constructs (Table 1).

Chondrocyte morphology within the regenerated tissue was analyzed because this characteristic indicates healthy non-pyknotic nuclei, chondrocyte shape and the quality of extracellular matrix. The chondrocytes of the Adhesive-cell-constructs and the Sutured-cell-constructs were similar to the Cell-constructs in morphology (Table 1).

The “Architecture within Entire Defect” classification in Table 1 is an assessment of the density of the regenerated tissue, which sometimes has a loose texture that appears as voids or clefts. The Adhesive-cell-constructs and the Sutured-cell-constructs were similar to the Cell-constructs in density of regenerated tissue.

In Table 1, the “Architecture of the surface at the defects” classification describes the ability of the surface of the regenerated tissue to withstand weight-bearing and joint-loading stresses. Most of the surface of the regenerated tissue within the Cell constructs was consistently covered with multi-layer tissue and extended to the superficial transitional zone of the adjacent cartilage. The architecture of the surface of the Adhesive-cell-constructs and of the Sutured-cell-constructs was similar to the Cell-constructs in grade (Table 1).

The “Penetration” classification in Table 1 describes edema formation in subchondral bone and is important to determine full recovery of the defects. Penetration of the Sutured-cell-constructs to subchondral bone was significantly greater than that of the Cell-constructs and the Adhesive-cell-constructs (P<0.05, Table 1).

The collagen type-II in the Adhesive-cell-constructs and in the Sutured-cell-constructs was similar in intensity to the Cell-constructs.

### Example 4

A human patient having with a cartilage defect, an injury to the cartilage, or a cartilage lesion in the knee undergoes surgery. If a cartilage lesion is not already present, such lesion may be created by removing cartilage from the site of the cartilage defect or the injury to the cartilage.

A first layer of a barrier composition comprising polyethylene glycol is introduced into the lesion and deposited at the bottom of the lesion, such as at the subchondral bone. The barrier composition is formulated so that it rapidly gels from a flowable liquid or paste to a load-bearing gel within 3 to 15 minutes. The barrier composition is allowed to cure or solidify so as to be effective to prevent entry and to block the migration of subchondral cells of the extraneous components, such as blood-borne agents, cell and cell debris, etc., into the cavity.

A support matrix is cut to match the dimensions of the cartilage lesion. The support matrix is then implanted into the cartilage lesion. No suturing is undertaken. At least one layer of sealant is added above the implanted support matrix. The wound is then sutured.

The patient is examined initially every two weeks to assess for pain and improvement in mobility.

The present invention is not to be limited in scope by the specific embodiments described herein. Indeed, various modifications of the invention in addition to those described herein will become apparent to those skilled in the art from the foregoing description and the accompanying figures. Such modifications are intended to fall within the scope of the appended claims. It is further to be understood that all values are approximate, and are provided for description.

Patents, patent applications, publications, product descriptions, and protocols are cited throughout this application, the disclosures of which are incorporated herein by reference in their entireties for all purposes.

1. A method for treating an injury or defect in articular cartilage, said method comprising:
a) preparing a matrix implant;
b) applying a barrier composition comprising a polymer to the bottom of the cartilage lesion; and
c) implanting said implant above the applied barrier composition.

2. The method of claim 1, wherein the barrier composition is applied to subchondral bone.

3. The method of claim 1, wherein the barrier composition is effective to block migration of cells, blood, or other material from the subchondral bone into the cartilage lesion.

4. The method of claim 1, wherein the matrix implant is an acellular matrix implant.

5. The method of claim 4, wherein the acellular matrix implant comprises one or more of a Type I collagen, a Type II collagen, a Type IV collagen, a collagen containing proteoglycan, a collagen containing glycosaminoglycan, a collagen containing glycoprotein, a polymer of an aromatic organic acid, gelatin, agarose, hyaluronan, fibronectin, laminin, a bioactive peptide growth factor, a cytokine, elastin, fibrin, a polymer made of polylactic acid, a polymer made of polyglycolic acid, poly(epsilon-caprolactone), a polyamino acid, a polypeptide gel, and a polymeric thermo-reversible gelling hydrogel (TRGH).

6. The method of claim 1, wherein said barrier composition comprises one or more of gelatin, Type I collagen, periodate-oxidized gelatin, photo-polymerizable polyethylene glycol-co-poly(α-hydroxy acid) diacrylate macromer, 4-armed polyethylene glycols derivatized with N-(acyloxy)succinimide and thiol plus methylated collagen, derivatized polyethylene glycol (PEG) cross-linked with alkylated collagen, tetra-N-hydroxysuccinimidyl, or tetra-thiol derivatized PEG, and cross-linked PEG with methylated collagen.

7. The method of claim 1, wherein said barrier composition comprises a sealant.

8. The method of claim 7, wherein the sealant forms a hydrogel after the barrier composition is applied to the subchondral bone.

9. The method of claim 1, wherein the barrier composition or the sealant comprises a polymer.

10. The method of claim 9, wherein the polymer is gelatin, polyethylene glycol (PEG), a derivatized PEG, a cyanoacrylate, a polyurethane, a poly(methylidene malonate), a derivatized polyvinyl alcohol, an acrylic polymer, fibrin, gelatin, polystyrene with catechol side chains, a polyester, a polymer secreted by Phragmatopoma californica, a copolymer of polyethylene glycol and polylactide, a copolymer of polyethylene glycol and polyglycolide, a polyether, a polysaccharide, an oxidized polysaccharide, a polycation polyamine, a polyanion, a poly(ester urea), a copolymer of polyethylene glycol and poly-lactide or poly-glycolide, 4-armed pentaerythritol thiol and a polyethylene glycol diacrylate, 4-armed tetra-N-hydroxysuccinimidyl ester or a tetra-thiol derivatized PEG, a polymer formed from gelatin and oxidized starch, a polymer formed from photo-polymerizable polyethylene glycol-co-poly(a-hydroxy acid) diacrylate macromers, periodate-oxidized gelatin, serum albumin and di-functional polyethylene glycol derivatized with maleimidyl, succinimidyl, phthalimidyl and related active groups, and 4-armed polyethylene glycols derivatized with succinimidyl ester and thiol, and methylated collagen.

11. The method of claim 10, wherein the polymer is gelatin or fibrin, and wherein the barrier composition comprises thrombin or a crosslinking agent.

12. The method of claim 1, wherein the barrier composition comprises a component that modulates viscosity.

13. The method of claim 1, wherein the barrier composition comprises a stabilizer.

14. The method of claim 1, wherein the barrier composition comprises an enzyme effective to increase the rate of degradation of the barrier composition.

15. The method of claim 1, wherein the barrier composition comprises a structural material.

16. The method of claim 15, wherein the structural material comprises one or more of a fiber, fibrin, alginate, hyaluronic acid, gelatin, cellulose, or collagen.

17. The method of claim 1, further comprising introducing a protective biodegradable polymer above the matrix implant.
